(19) United States
(12) Patent Application Publication
(10) Pub. No.: US 2024/0049942 A1
(43) Pub. Date: Feb. 15, 2024

(54) ENDOSCOPE SYSTEM AND METHOD OF OPERATING THE SAME

(71) Applicant: FUJIFILM Corporation, Tokyo (JP)

(72) Inventors: Masato YOSHIOKA, Kanagawa (JP); Takeshi FUKUDA, Kanagawa (JP)

(73) Assignee: FUJIFILM Corporation, Tokyo (JP)

(21) Appl. No.: 18/490,785

(22) Filed: Oct. 20, 2023

Related U.S. Application Data
(63) Continuation of application No. PCT/JP2022/017485, filed on Apr. 11, 2022.

(30) Foreign Application Priority Data
Apr. 23, 2021 (JP) 2021-073355

(51) Int. Cl. A61B 1/00; A61B 1/05

(52) U.S. Cl. CPC A61B 1/00009; A61B 1/00045; A61B 1/00147; A61B 1/05; A61B 1/00059

(57) ABSTRACT

A region of interest is detected from a subject image. In a case where a position of the region of interest ROI in the subject image is included in a specific region, a size of the region of interest ROI is estimated. In a case where a position of at least the region of interest ROI is not included in the specific region, the size of the region of interest ROI is not estimated.

### CROSS-REFERENCE TO RELATED APPLICATIONS

This application is a Continuation of PCT International Application No. PCT/JP2022/017485 filed on 11 Apr. 2022, which claims priority under 35 U.S.C § 119(a) to Japanese Patent Application No. 2021-073355 filed on 23 Apr. 2021. The above application is hereby expressly incorporated by reference, in its entirety, into the present application.

### BACKGROUND OF THE INVENTION

### 1. Field of the Invention

The present invention relates to an endoscope system that estimates the size of a region of interest, such as a lesion area, and a method of operating the endoscope system.

### 2. Description of the Related Art

In the field of endoscopy and the like, the size of a region of interest, such as a found lesion area, is important information as one of criteria used to determine a diagnosis method or a treatment method. However, it is difficult to visually estimate the size of the region of interest due to problems, such as distortion peculiar to an image obtained from an endoscope or no landmark of an existing size. Accordingly, in US2020/0279373A1, the size of a region of interest can be estimated with reference to the size of a treatment tool that is displayed simultaneously with the region of interest.

### SUMMARY OF THE INVENTION

In recent years, the estimation of a size using artificial intelligence (AI) has been proposed. However, there are the following problems in the field of endoscopy in a case where a size is estimated. Since a wide-angle lens, which can be used to make an observation with a wide visual field, is used as an imaging optical system of an endoscope, an image is greatly distorted in a case where a close-up observation is made, a case where an object to be observed is displayed at an end of an endoscopic image, and the like. For this reason, even though the same object is imaged, the size of the object is displayed differently depending on an observation distance, disposition, or the like. As a result, a large error may occur in a case where the size of the object is estimated.

An object of the present invention is to provide an endoscope system that can estimate a size of a region of interest with high accuracy in a case where the region of interest is detected from an image, and a method of operating the endoscope system.

An endoscope system according to an aspect of the present invention comprises a processor; and the processor detects a region of interest from a subject image, and performs a control to estimate a size of the region of interest in a case where a position of the region of interest in the subject image is included in a specific region and not to estimate the size in a case where the position of at least the region of interest is not included in the specific region.

It is preferable that the processor sets a position, a size, or a range of the specific region using optical information included in an imaging optical system used for acquisition of the subject image. It is preferable that the processor receives endoscope information about an endoscope, and specifies the optical information from the endoscope information. It is preferable that the processor sets a position, a size, or a range of the specific region using an observation distance indicating a distance to the region of interest. It is preferable that the endoscope system further comprises an endoscope emitting distance-measuring laser such that the distance-measuring laser intersect with an optical axis of an imaging optical system used for acquisition of the subject image, and the processor measures the observation distance from an irradiation position of the distance-measuring laser in the subject image. It is preferable that the processor sets a position, a size, or a range of the specific region using optical information included in an imaging optical system used for acquisition of the subject image and an observation distance indicating a distance to the region of interest.

It is preferable that the processor notifies a user of detection of the region of interest or the size of the region of interest. It is preferable that the processor gives a movement guidance notification notifying a user of a direction in which the region of interest is to be moved to be included in the specific region in a case where the position of the region of interest is not included in the specific region.

It is preferable that, in a case where the position of the region of interest is not included in the specific region or in a case where the size of the region of interest is larger than a size of the specific region and the size is not estimated, the processor gives a non-estimable notification notifying that the size is not capable of being estimated. It is preferable that the non-estimable notification is given using a display in the subject image or a voice.

It is preferable that the specific region is included in a region that is within a range of a certain distance from a center of the subject image. It is preferable that, in a case where a first axis extending in a first direction and a second axis extending in a second direction orthogonal to the first direction are defined in the subject image, the specific region is a rectangular region surrounded by a first lower limit boundary line indicating a lower limit on the first axis, a first upper limit boundary line indicating an upper limit on the first axis, a second lower limit boundary line indicating a lower limit on the second axis, and a second upper limit boundary line indicating an upper limit on the second axis. It is preferable that the specific region is a circular or oval region. It is preferable that the processor displays the specific region on a display.

A method of operating an endoscope system including a processor according to another aspect of the present invention comprises: a step of detecting a region of interest from a subject image; and a step of performing a control to estimate a size of the region of interest in a case where a position of the region of interest in the subject image is included in a specific region and not to estimate the size in a case where the position of at least the region of interest is not included in the specific region.

According to the present invention, in a case where a region of interest is detected from an image, it is possible to estimate the size of the region of interest with higher accuracy than in the related art.

### BRIEF DESCRIPTION OF THE DRAWINGS

FIG. 1 is a schematic diagram of an endoscope system.

FIG. 2 is a block diagram showing the functions of the endoscope system.

(A) of FIG. 3 is an image diagram showing a state where a digital zoom function is turned off and (B) of FIG. 3 is an image diagram showing a state where a digital zoom function is turned on.

FIG. 4 is a block diagram showing the functions of a signal processing unit.

FIG. 5 is a diagram illustrating region notification information to be displayed in a case where a region of interest is detected.

FIG. 6 is an image diagram showing size information.

FIG. 7 is an image diagram showing a specific region.

FIG. 8 is an image diagram in a case where the position of the region of interest is included in the specific region.

FIG. 9 is an image diagram in a case where the position of the region of interest is not included in the specific region.

FIG. 10 is a diagram illustrating a method of reading out endoscope information.

FIG. 11A is a diagram illustrating a method of setting a specific region from first optical information, and FIG. 11B is a diagram illustrating a method of setting a specific region from second optical information.

FIG. 12A is a diagram illustrating a method of setting a specific region from a first observation distance, FIG. 12B is a diagram illustrating a method of setting a specific region from a second observation distance, and FIG. 12C is a diagram illustrating a method of setting a specific region from a third observation distance.

FIG. 13 is a diagram illustrating a method of acquiring an observation distance.

FIG. 14 is a diagram illustrating the irradiation of distance-measuring laser.

FIG. 15 is an image diagram showing a movement guidance direction.

FIG. 16 is an image diagram displaying a message for guiding a region of interest to a specific region.

FIG. 17 is an image diagram showing a non-estimable notification outside a size-estimable region.

FIG. 18 is an image diagram showing a non-estimable notification caused by a size that cannot be estimated.

FIG. 19 is a flowchart showing a series of flows of a length measurement mode.

FIG. 20 is an image diagram in a case where a specific region is an oval region.

### DESCRIPTION OF THE PREFERRED EMBODIMENTS

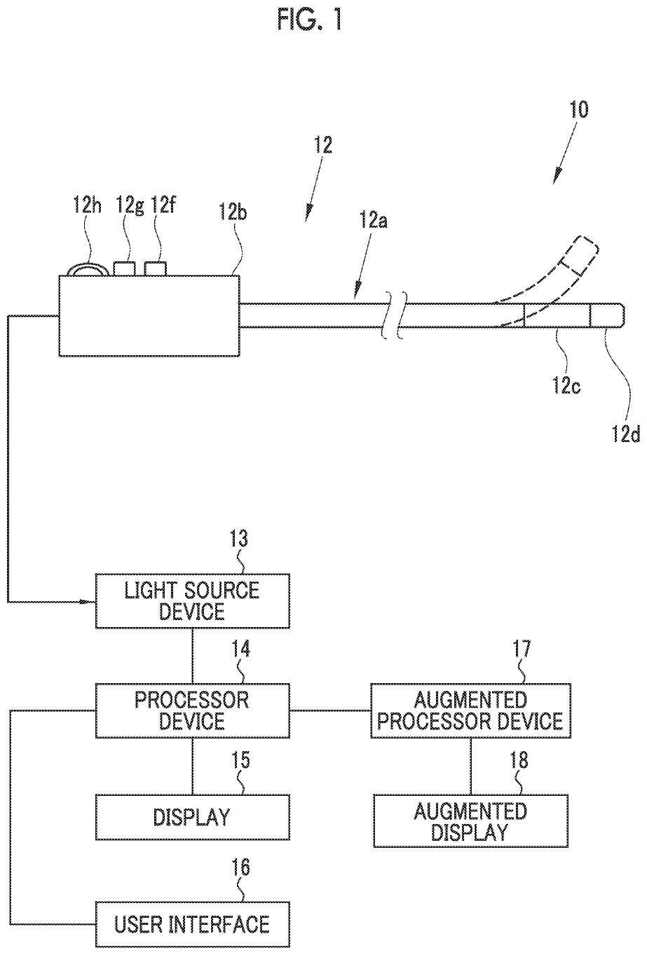

As shown in FIG. 1, an endoscope system 10 includes an endoscope 12, a light source device 13, a processor device 14, a display 15, a user interface 16, an augmented processor device 17, and an augmented display 18. The endoscope 12 is optically connected to the light source device 13, and is electrically connected to the processor device 14. The endoscope 12 includes an insertion part 12a that is to be inserted into a body of an object to be observed, an operation part 12b that is provided at a proximal end portion of the insertion part 12a, and a bendable part 12c and a distal end part 12d that are provided on a distal end side of the insertion part 12a. In a case where the operation part 12b is operated, the bendable part 12c is operated to be bent. As the bendable part 12c is operated to be bent, the distal end part 12d is made to face in a desired direction.

Further, the operation part 12b is provided with an observation mode selector switch 12f that is used for an operation for switching an observation mode, a static image-acquisition instruction switch 12g that is used to give an instruction to acquire a static image of the object to be observed, and a zoom operation part 12h that is used for an operation of a zoom lens 21b. Meanwhile, in a case where the zoom lens 21b is not provided, the zoom operation part 12h is also not provided.

The processor device 14 is electrically connected to the display 15 and the user interface 16. The display 15 outputs and displays an image, information, or the like of the object to be observed that is processed by the processor device 14. The user interface 16 includes a keyboard, a mouse, a touch pad, a microphone, and the like and has a function to receive an input operation, such as function settings. The augmented processor device 17 is electrically connected to the processor device 14. The augmented display 18 outputs and displays an image, information, or the like that is processed by the augmented processor device 17.

The endoscope 12 has a normal observation mode, a special light observation mode, and a length measurement mode. The normal observation mode and the special light observation mode are switched by the observation mode selector switch 12f The length measurement mode can be executed in either the normal observation mode or the special light observation mode, and ON and OFF of the length measurement mode can be switched by a selector switch (not shown) provided in the user interface 16 separately from the observation mode selector switch 12f The normal observation mode is a mode in which an object to be observed is illuminated with illumination light. The special light observation mode is a mode in which an object to be observed is illuminated with special light different from the illumination light. In the length measurement mode, in a case where a region of interest, such as a lesion area, is detected in an object to be observed, the size of the region of interest is estimated and the estimated size of the region of interest is displayed on the augmented display 18.

In the length measurement mode, an object to be observed is illuminated with illumination light or special light. The illumination light is light that is used to apply brightness to the entire object to be observed to observe the entire object to be observed. The special light is light that is used to highlight a specific region of the object to be observed.

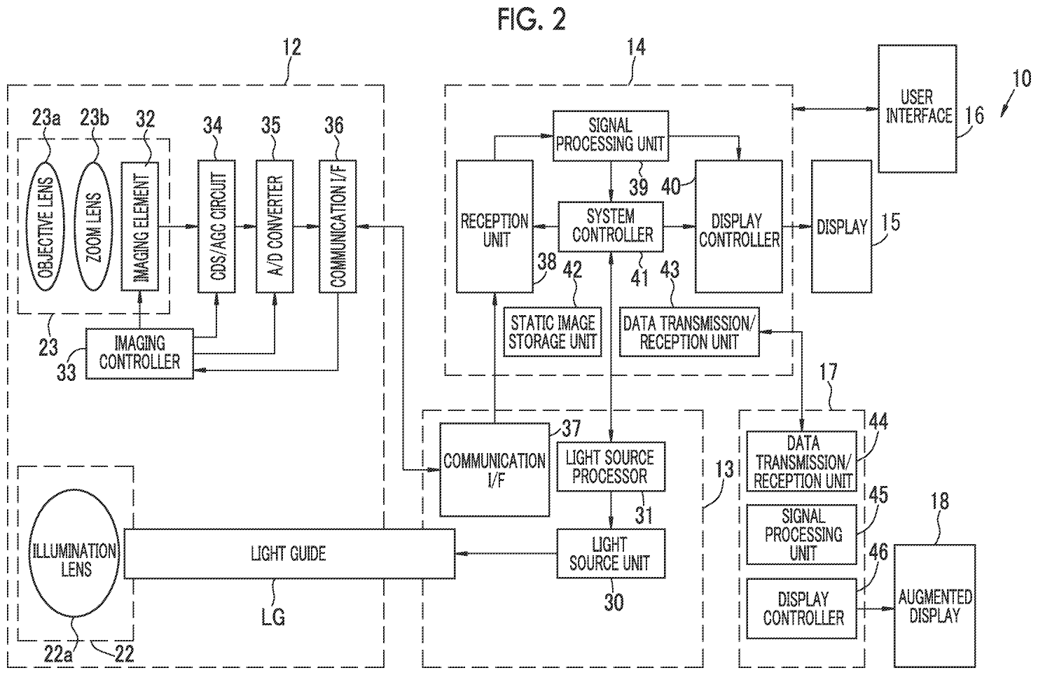

In a case where the static image-acquisition instruction switch 12g is operated by a user, the screen of the display 15 is frozen and displayed and an alert sound (for example, “beep”) informing the acquisition of a static image is generated together. Then, the static images of the subject image, which are obtained before and after the operation timing of the static image-acquisition instruction switch 12g, are stored in a static image storage unit 42 (see FIG. 2) provided in the processor device 14. The static image storage unit 42 is a storage unit, such as a hard disk or a universal serial bus (USB) memory. In a case where the processor device 14 can be connected to a network, the static images of the subject image may be stored in a static image storage server (not shown), which is connected to the network, instead of or in addition to the static image storage unit 42.

A static image-acquisition instruction may be given using an operation device other than the static image-acquisition instruction switch 12g. For example, a foot pedal may be connected to the processor device 14, and may be adapted to give a static image-acquisition instruction in a case where a user operates the foot pedal (not shown) with a foot. A static image-acquisition instruction may also be given by a foot pedal that is used to switch a mode. Further, a gesture recognition unit (not shown), which recognizes the gestures of a user, may be connected to the processor device 14, and may be adapted to give a static image-acquisition instruction in a case where the gesture recognition unit recognizes a specific gesture of a user. The gesture recognition unit may also be used to switch a mode.

Further, a sight line input unit (not shown), which is provided close to the display 15, may be connected to the processor device 14, and may be adapted to give a static image-acquisition instruction in a case where the sight line input unit recognizes that a user's sight line is in a predetermined region of the display 15 for a predetermined time or longer. Furthermore, a voice recognition unit (not shown) may be connected to the processor device 14, and may be adapted to give a static image-acquisition instruction in a case where the voice recognition unit recognizes a specific voice generated by a user. The voice recognition unit may also be used to switch a mode. Moreover, an operation panel (not shown), such as a touch panel, may be connected to the processor device 14, and may be adapted to give a static image-acquisition instruction in a case where a user performs a specific operation on the operation panel. The operation panel may also be used to switch a mode.

As shown in FIG. 2, the light source device 13 comprises a light source unit 30 and a light source processor 31. The light source unit 30 generates the illumination light or the special light that is used to illuminate the subject. The illumination light or the special light, which is emitted from the light source unit 30, is incident on a light guide LG, and the subject is irradiated with the illumination light or the special light through an illumination lens 22a included in an illumination optical system 22. A white light source emitting white light, a plurality of light sources, which include a white light source and a light source emitting another color light (for example, a blue light source emitting blue light), or the like is used as a light source of the illumination light in the light source unit 30. Further, a light source, which emits broadband light including blue narrow-band light used to highlight superficial information about superficial blood vessels and the like, is used as a light source of the special light in the light source unit 30. Light (for example, white light, special light, or the like) in which at least one of violet light, blue light, green light, or red light is combined may be used as the illumination light.

The light source processor 31 controls the light source unit 30 on the basis of an instruction given from a system controller 41 of the processor device 14. The system controller 41 gives an instruction related to light source control to the light source processor 31. In the case of the normal observation mode, the system controller 41 performs a control to turn on the illumination light. In the case of the special light observation mode, the system controller 41 performs a control to turn on the special light. In the case of the length measurement mode, the system controller 41 performs a control to turn on the illumination light or the special light.

An imaging optical system 23 includes an objective lens 23a, a zoom lens 23b, and an imaging element 32. Light reflected from the object to be observed is incident on the imaging element 32 via the objective lens 23a and the zoom lens 23b. Accordingly, the reflected image of the object to be observed is formed on the imaging element 32. The imaging optical system 23 may not be provided with the zoom lens 23b.

The zoom lens 23b has an optical zoom function to enlarge or reduce the subject by moving between a telephoto end and a wide end as a zoom function. ON and OFF of the optical zoom function can be switched by the zoom operation part 12h (see FIG. 1) provided on the operation part 12b of the endoscope, and the subject is enlarged or reduced at a specific magnification ratio in a case where the zoom operation part 12h is further operated in a state where the optical zoom function is turned on. In a case where the zoom lens 23b is not provided, the optical zoom function is not provided.

The imaging element 32 is a color image pickup sensor, and picks up the reflected image of an object to be examined and outputs image signals. It is preferable that the imaging element 32 is a charge coupled device (CCD) image pickup sensor, a complementary metal-oxide semiconductor (CMOS) image pickup sensor, or the like. The imaging element 32 used in the present invention is a color image pickup sensor that is used to obtain red images, green images, and red images corresponding to three colors of R (red), G (green), and B (blue). The red image is an image that is output from red pixels provided with red color filters in the imaging element 32. The green image is an image that is output from green pixels provided with green color filters in the imaging element 32. The blue image is an image that is output from blue pixels provided with blue color filters in the imaging element 32. The imaging element 32 is controlled by an imaging controller 33.

Image signals output from the imaging element 32 are transmitted to a CDS/AGC circuit 34. The CDS/AGC circuit 34 performs correlated double sampling (CDS) or auto gain control (AGC) on the image signals that are analog signals. The image signals, which have been transmitted through the CDS/AGC circuit 34, are converted into digital image signals by an analog/digital converter (A/D converter) 35. The digital image signals, which have been subjected to A/D conversion, are input to a communication interface (I/F) 37 of the light source device 13 through a communication interface (I/F) 36.

In the processor device 14, programs related to various types of processing, control, or the like are incorporated into a program storage memory (not shown). The system controller 41 formed of a processor of the processor device 14 operates the programs incorporated into the program storage memory, so that the functions of a reception unit 38 connected to the communication interface (I/F) 37 of the light source device 13, a signal processing unit 39, and a display controller 40 are realized.

The reception unit 38 receives the image signals, which are transmitted from the communication I/F 37, and transmits the image signals to the signal processing unit 39. A memory, which temporarily stores the image signals received from the reception unit 38, is built in the signal processing unit 39, and the signal processing unit 39 processes an image signal group, which is a set of the image signals stored in the memory, to generate the subject image. The reception unit 38 may directly transmit control signals, which are related to the light source processor 31, to the system controller 41.

In a case where the endoscope 12 is set to the normal observation mode, signal assignment processing for assigning the blue image of the subject image to B channels of the display 15, assigning the green image of the subject image to G channels of the display 15, and assigning the red image of the subject image to R channels of the display 15 is performed in the signal processing unit 39. As a result, a color subject image is displayed on the display 15. The same signal assignment processing as that in the normal observation mode is performed even in the length measurement mode.

On the other hand, in a case where the endoscope 12 is set to the special light observation mode, the red image of the subject image is not used for the display of the display 15, the blue image of the subject image is assigned to the B channels and the G channels of the display 15, and the green image of the subject image is assigned to the R channels of the display 15 in the signal processing unit 39. As a result, a pseudo-color subject image is displayed on the display 15.

Further, in a case where the endoscope 12 is set to the length measurement mode, the signal processing unit 39 transmits a subject image to a data transmission/reception unit 43. The data transmission/reception unit 43 transmits data, which are related to the subject image, to the augmented processor device 17. The data transmission/reception unit 43 can receive data and the like from the augmented processor device 17. The received data can be processed by the signal processing unit 39 or the system controller 41.

In a case where a digital zoom function is set to ON as a zoom function by the user interface 16, the signal processing unit 39 cuts out a portion of the subject image and enlarges or reduces the cut portion. As a result, the subject is enlarged or reduced at a specific magnification. (A) of FIG. 3 shows a subject image obtained in a state where the digital zoom function is turned off and (B) of FIG. 3 shows a subject image obtained in a state where the digital zoom function is turned on so that a central portion of the subject image shown in (A) of FIG. 3 is cut out and enlarged. In a case where the digital zoom function is turned off, the enlargement or reduction of the subject using the cutout of the subject image is not performed.

The display controller 40 causes the display 15 to display the subject image that is generated by the signal processing unit 39. The system controller 41 performs various controls on the endoscope 12, the light source device 13, the processor device 14, and the augmented processor device 17. The system controller 41 performs the control of the imaging element 32 via the imaging controller 33 provided in the endoscope 12. The imaging controller 33 also performs the control of the CDS/AGC circuit 34 and the A/D converter 35 in accordance with the control of the imaging element 32.

The augmented processor device 17 receives data, which are transmitted from the processor device 14, by a data transmission/reception unit 44. The subject image is included in the data received by the data transmission/reception unit 44. A signal processing unit 45 performs processing related to the length measurement mode on the basis of the data that are received by the data transmission/reception unit 44. Specifically, in a case where a region of interest is detected from the subject image, the signal processing unit 45 performs processing of estimating the size of the region of interest and superimposing and displaying the estimated size of the region of interest on the subject image. In a case where a region of interest is not detected, the display controller 46 causes the augmented display 18 to display the subject image. In a case where a region of interest is detected, the display controller 46 causes the augmented display 18 to display the subject image on which the size of the region of interest is superimposed and displayed. The data transmission/reception unit 44 can transmit data and the like to the processor device 14.

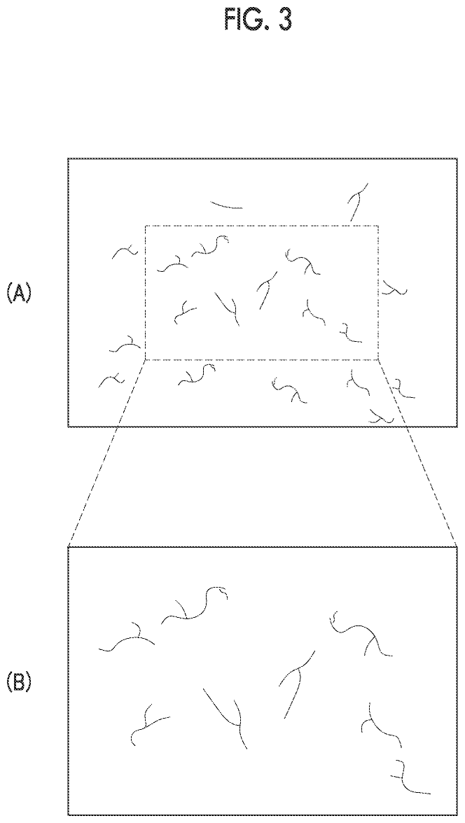

As shown in FIG. 4, the signal processing unit 45 comprises a region-of-interest detector 50, a size estimation unit 51, a first notification unit 52, a size estimation controller 53, a specific region setting unit 54, an optical information acquisition unit 55, an observation distance acquisition unit 56, and a second notification unit 57.

In the augmented processor device 17, programs related to various types of processing, control, or the like are incorporated into a program storage memory (not shown). A central controller (not shown) formed of a processor of the augmented processor device 17 operates the programs incorporated into the program storage memory, so that the functions of the region-of-interest detector 50, the size estimation unit 51, the first notification unit 52, the size estimation controller 53, the specific region setting unit 54, the optical information acquisition unit 55, the observation distance acquisition unit 56, and the second notification unit 57 are realized.

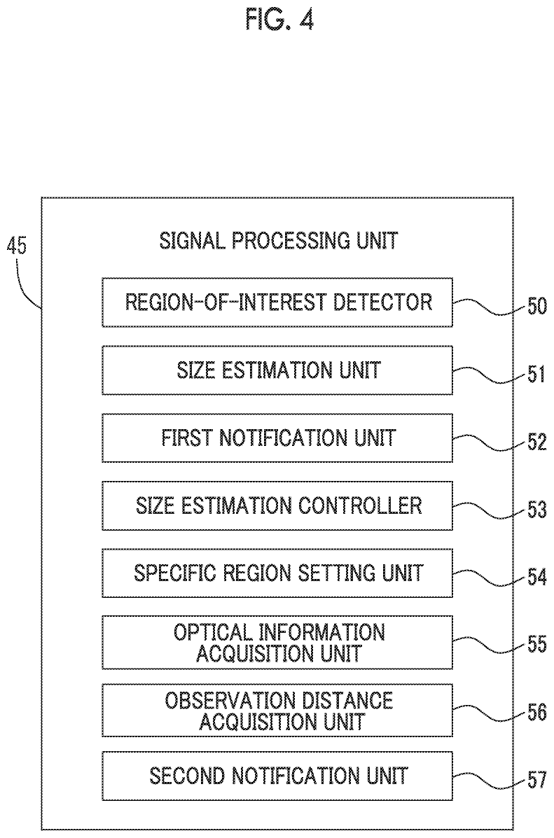

The region-of-interest detector 50 detects a region of interest from a subject image. In a case where a region of interest ROI is detected in a subject image PS, region notification information 61 showing that the region of interest ROI is present is displayed around the region of interest ROI on the augmented display 18 by the first notification unit 52 as shown in FIG. 5. In FIG. 5, a rectangular bounding box is used as the region notification information 61.

It is preferable that processing performed by a region-of-interest detection-learning model obtained from learning using, for example, a neural network (NN), a convolutional neural network (CNN), Adaboost, or random forest is used as region-of-interest detection processing performed by the region-of-interest detector 50. That is, it is preferable that the detection of a region of interest, such as a lesion area, is output from the region-of-interest detection-learning model in a case where the subject image is input to the region-of-interest detection-learning model. Further, as the region-of-interest detection processing, the detection of a region of interest may be performed on the basis of a feature quantity (parameters) that is obtained from color information of the subject image, a gradient of pixel values, or the like. The gradient of pixel values, or the like is changed depending on, for example, the shape (the overall undulation, local depressions or bumps, or the like of a mucous membrane), the color (a color, such as whitening caused by inflammation, bleeding, redness, or atrophy), the characteristics of a tissue (the thickness, depth, or density of a blood vessel, a combination thereof, or the like), the characteristics of structure (a pit pattern, and the like), or the like of a subject.

Further, the region of interest detected by the region-of-interest detection processing is a region including, for example, a lesion area typified by a cancer, a treatment trace, a surgical scar, a bleeding site, a benign tumor area, an inflammation area (including a portion with changes, such as bleeding or atrophy, in addition to so-called inflammation), a cauterization scar caused by heating or a marking area marked by coloring with a coloring agent, a fluorescent drug, or the like, or a biopsy area where biopsy examination (so called a biopsy) is performed. That is, a region including a lesion, a region having a possibility of a lesion, a region where certain treatment, such as a biopsy, has been performed, a treatment tool, such as clips or forceps, a region which is required to be observed in detail regardless of a possibility of a lesion, such as a dark region (the back of folds, a region where observation light is difficult to reach due to the depth of the lumen), or the like may be a region of interest. The region-of-interest detection processing detects a region including at least one of a lesion area, a treatment trace, a surgical scar, a bleeding site, a benign tumor area, an inflammation area, a marking area, or a biopsy area, as the region of interest.

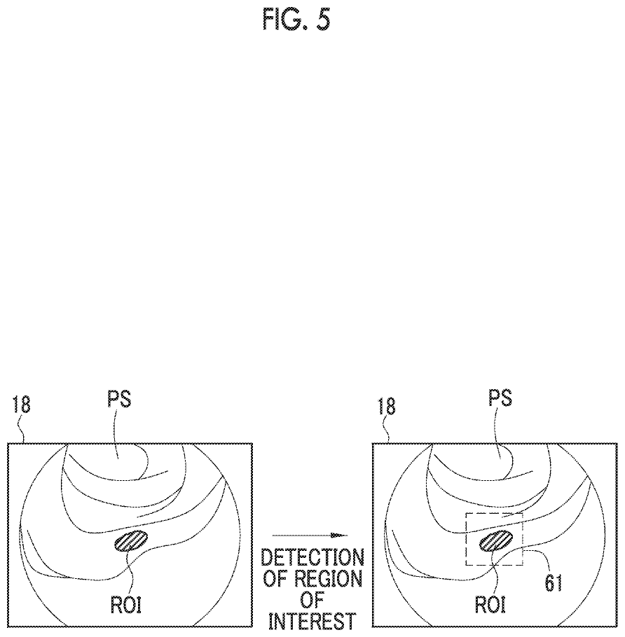

The size estimation unit 51 estimates the size of the region of interest. In a case where the size of the region of interest has been estimated, size information 62 is displayed near the region of interest ROI on the augmented display 18 by the first notification unit 52 as shown in FIG. 6. For example, “numerical value+unit for size”, such as “5 mm”, is represented as the size information.

It is preferable that processing performed by a size estimation-learning model obtained from learning using, for example, a neural network (NN), a convolutional neural network (CNN), Adaboost, or random forest is used as size estimation processing performed by the size estimation unit 51. That is, it is preferable that size information about the region of interest is output from the size estimation-learning model in a case where the subject image including the region of interest is input to the size estimation-learning model. Further, as the size estimation processing, a size may be estimated on the basis of a feature quantity (parameters) that is obtained from color information of the subject image, a gradient of pixel values, or the like.

In a case where the position of the region of interest in the subject image is included in a specific region, the size estimation controller 53 performs a control to estimate a size. In a case where the position of the region of interest is not included in the specific region, the size estimation controller 53 performs a control not to estimate a size. The inside of the specific region is a region where a size can be estimated with certain accuracy, and the outside of the specific region is a region where it is difficult to estimate a size with certain accuracy.

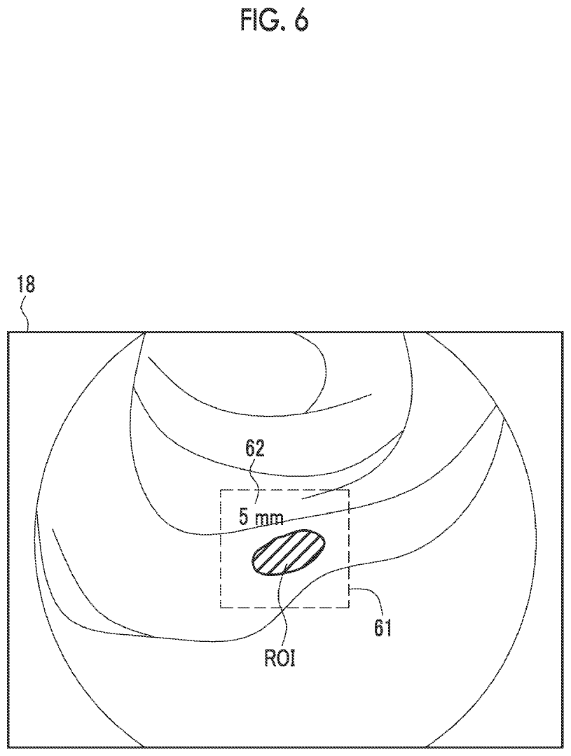

In a case where an X axis (first axis) extending in an X direction (first direction) and a Y axis (second axis) extending in a Y direction (a second direction orthogonal to the first direction) are defined in the subject image, a specific region 64 is a rectangular region that is surrounded by a first lower limit boundary line X1 indicating a lower limit on the X axis, a first upper limit boundary line X2 indicating an upper limit on the X axis, a second lower limit boundary line Y1 indicating a lower limit on the Y axis, and a second upper limit boundary line Y2 indicating an upper limit on the Y axis as shown in FIG. 7.

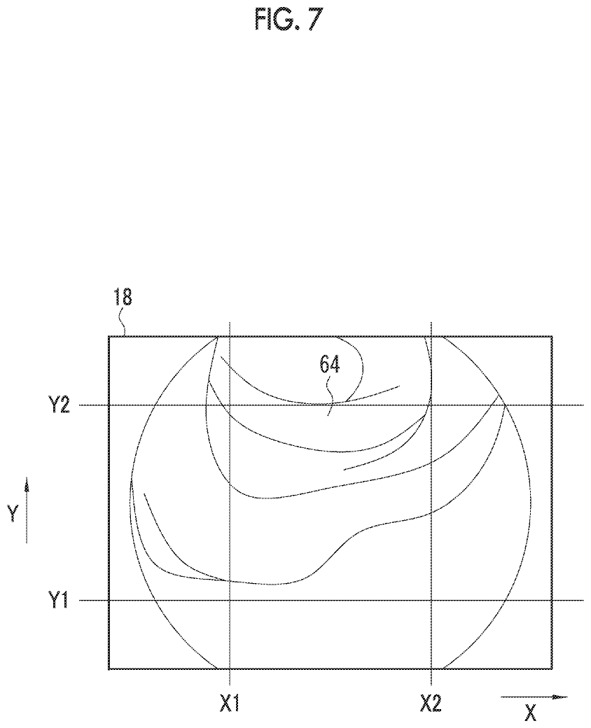

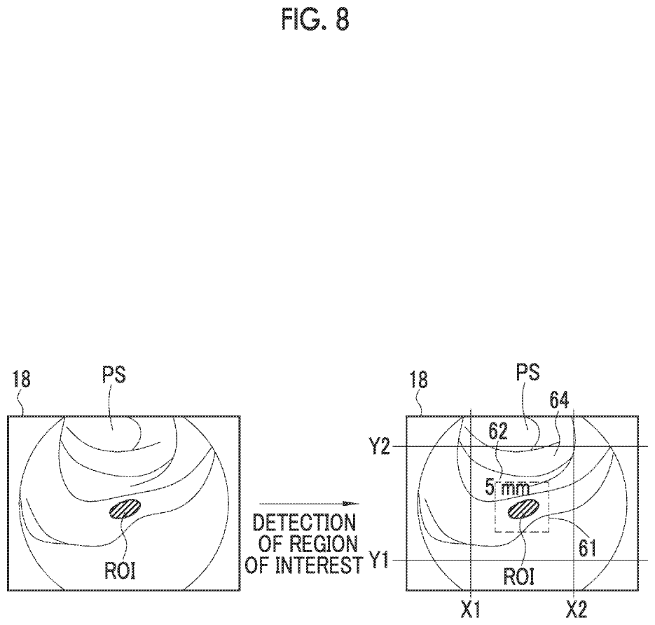

In a case where the position (Xat, Yat) of the region of interest ROI is present inside the specific region 64 (X1≤Xat≤X2 and Y1≤Yat≤Y2) as shown in FIG. 8, the size estimation controller 53 estimates a size. On the other hand, in a case where the position of the region of interest ROI is present outside the specific region 64 (Xat<X1, X2<Xat, and Yat<Y1, Y2<Yat) as shown in FIG. 9, the size estimation controller 53 does not estimate a size.

The specific region setting unit 54 sets the position, the size, or the range of the specific region. Specifically, the specific region setting unit 54 sets the position, the size, or the range of the specific region using optical information that is included in the imaging optical system 23 used for the acquisition of the subject image. For example, the objective lens 23a and the zoom lens 23b are included in the optical information. Since the aberration of the objective lens 23a or the zoom lens 23b has a variation for each imaging optical system 23, distortion or the like in the shape of a peripheral image of the subject image also has a variation for each imaging optical system 23. For this reason, the position, the size, or the range of the specific region 64, which determines the accuracy in the estimation of the size of the region of interest, also varies for each imaging optical system 23. The zoom magnification of the digital zoom function may be included in the optical information. In this case, the specific region setting unit 54 may set the position, the size, or the range of the specific region depending on the zoom magnification of the digital zoom function.

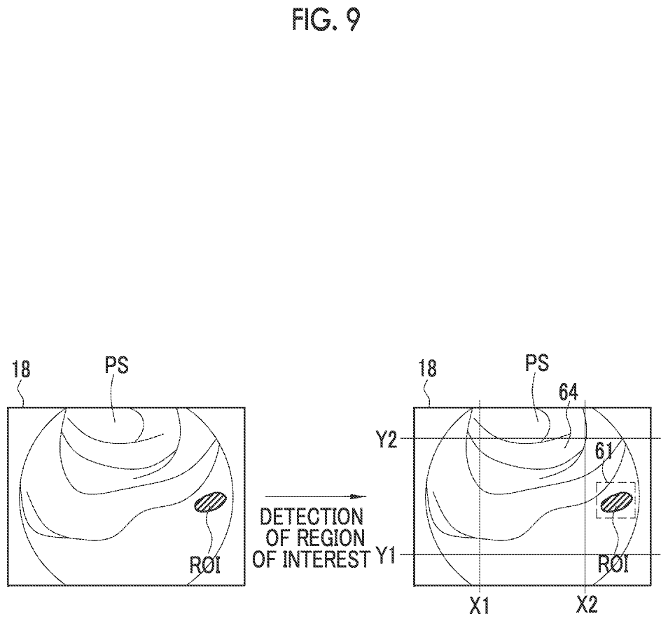

The specific region setting unit 54 specifies the optical information from endoscope information about the endoscope 12. Model information and the like of the endoscope 12 are included in the endoscope information. As shown in FIG. 10, the endoscope information is stored in an endoscope information storage memory 65 of the endo scope 12. An endoscope information acquisition unit 66 provided in the processor device 14 reads out the endoscope information from an endoscope information storage memory 63 of the endoscope 12. The read endoscope information is transmitted to the specific region setting unit 54 of the augmented processor device 17.

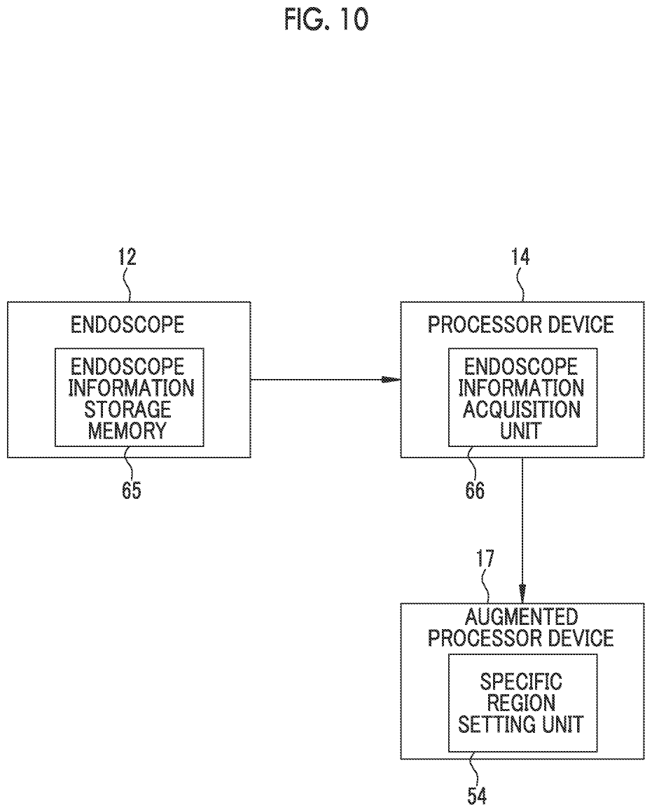

The specific region setting unit 54 includes an optical information table (not shown) in which endoscope information and optical information are associated with each other, and specifies optical information corresponding to the endoscope information, which is received from the processor device 14, with reference to the optical information table. For example, a specific region 64a having a first size is set as shown in FIG. 11A in a case where the optical information is first optical information, and a specific region 64b having a second size different from the first size is set as shown in FIG. 11B in a case where the optical information is second optical information. Here, since distortion in the shape of the peripheral portion of an image in the case of the second optical information is larger than that in the case of the first optical information, the specific region 64b having the second size smaller than the first size is set in the case of the second optical information. In a case where optical information is included in endoscope information, a specific region may be set on the basis of the endoscope information (in this case, the optical information table is unnecessary).

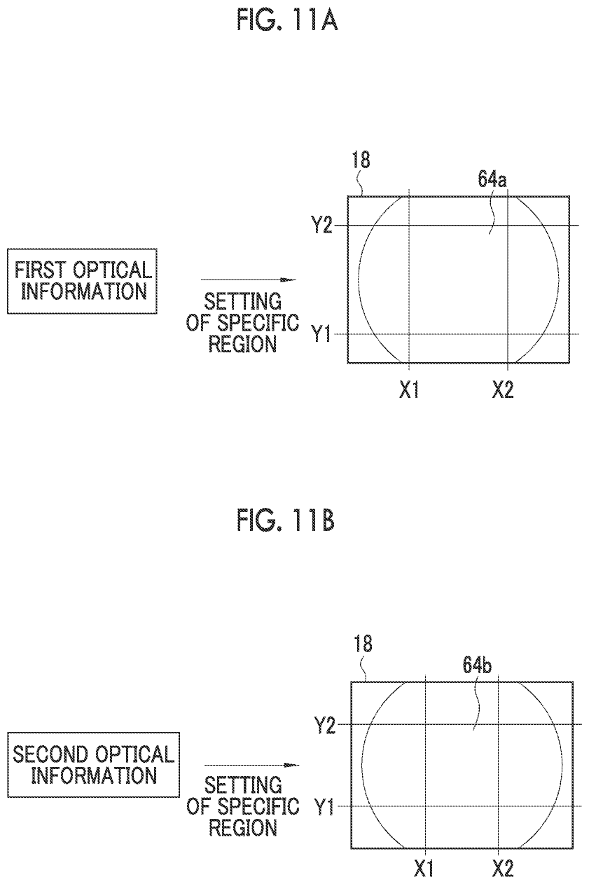

Further, the specific region setting unit 54 may set the position, the size, or the range of the specific region using an observation distance that indicates a distance to the region of interest. It is preferable that the observation distance is a distance between the distal end part 12d of the endoscope 12 and the region of interest. For example, in a case where the observation distance is a first observation distance of a distant view, a specific region 64c having a first size is set as shown in FIG. 12A. Further, in a case where the observation distance is a second observation distance between a distant view and the near view, a specific region 64d having a second size smaller than the first size is set as shown in FIG. 12B. Furthermore, in a case where the observation distance is a third observation distance of a near view, a specific region 64e having a third size smaller than the second size is set as shown in FIG. 12C. The reason for this is that distortion in the shape of the peripheral portion of an image is increased as the observation distance is reduced. Accordingly, the size of a specific region is also set to be reduced. The specific region setting unit 54 may set the position, the size, or the range of the specific region using both the optical information and the observation distance included in the imaging optical system 23.

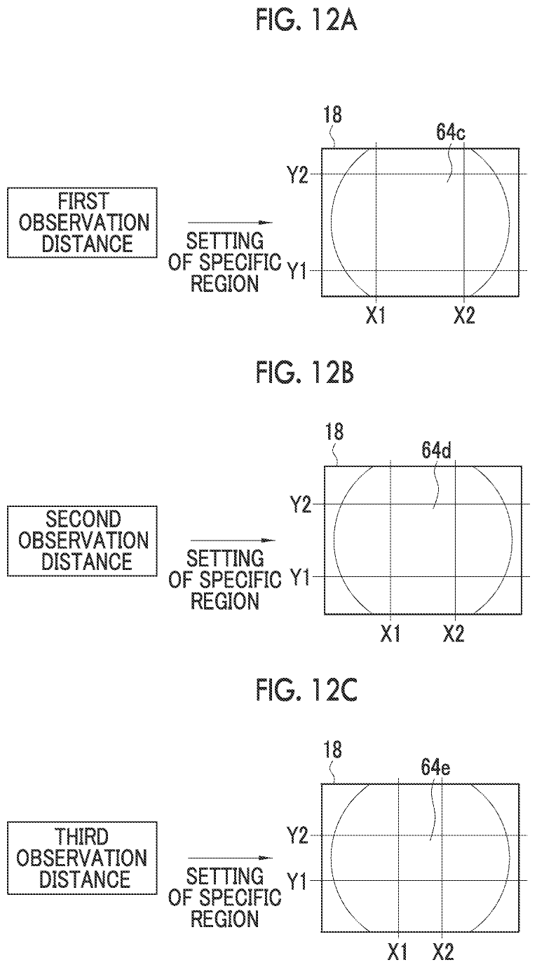

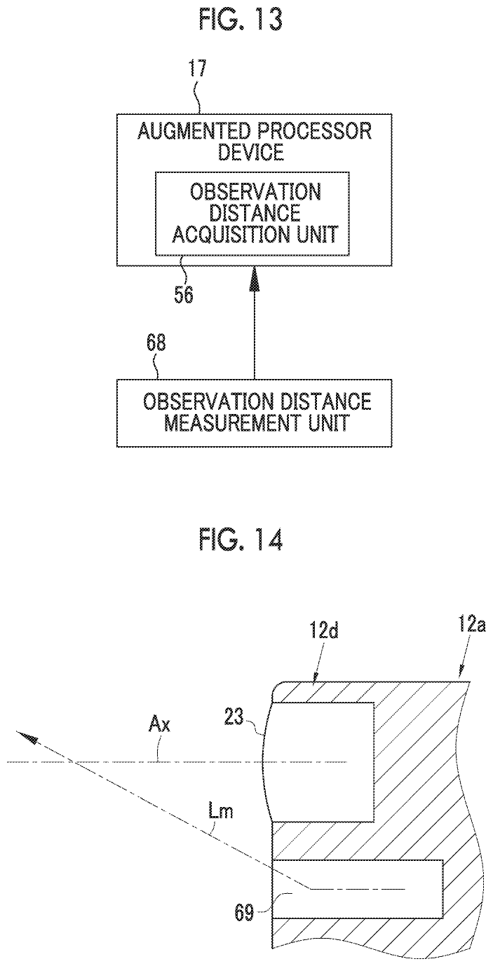

The observation distance is acquired by the observation distance acquisition unit 56. As shown in FIG. 13, the observation distance acquisition unit 56 acquires an observation distance using an observation distance measurement unit 68 provided in the endoscope 12 or the processor device 14. The acquired observation distance is transmitted to the observation distance acquisition unit 56 of the augmented display 18. It is preferable that the observation distance measurement unit 68 is, for example, a stereo camera, Time Of Flight (TOF) camera, an ultrasound device, a forceps device, or the like. Further, distance-measuring laser Lm may be emitted to intersect with an optical axis Ax of the imaging optical system 23 of the endoscope as shown in FIG. 14, and an observation distance may be measured from an irradiation position of the distance-measuring laser Lm in the subject image. This method of measuring the observation distance uses that the irradiation position of the distance-measuring laser Lm is changed depending on a change in the observation distance as the distance-measuring laser Lm is emitted. The distance-measuring laser Lm is emitted from a distance-measuring laser emitting unit 69.

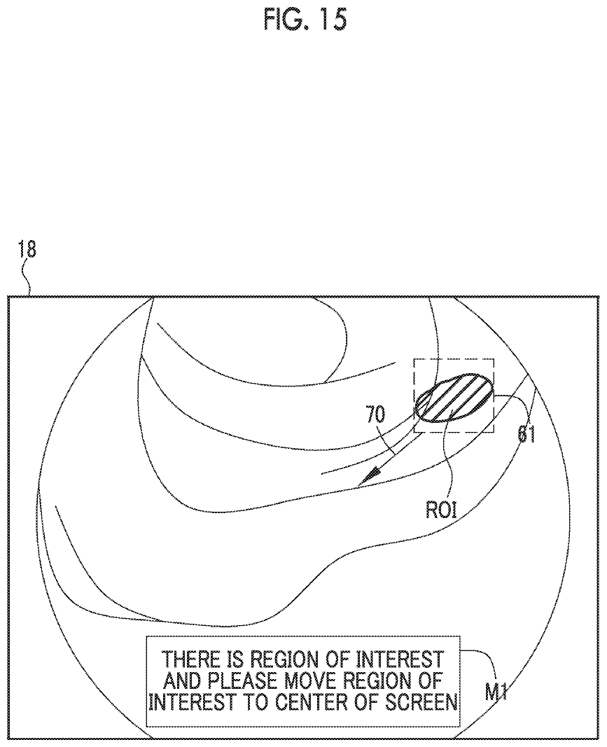

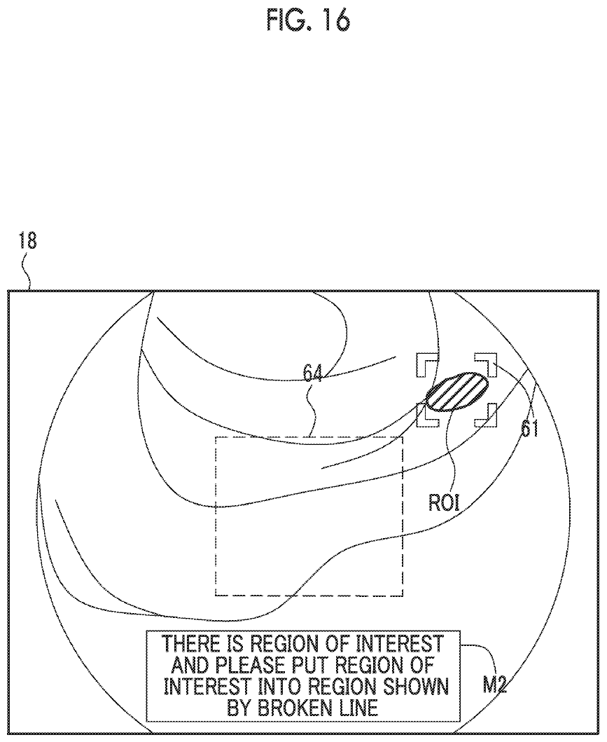

In a case where the position of the region of interest is not included in the specific region, the second notification unit 57 gives a movement guidance notification notifying a user of a direction in which the region of interest is to be moved to be included in the specific region. Specifically, as shown in FIG. 15, a movement guidance direction 70 toward the specific region may be displayed on the region notification information 61 as movement notification information. In this case, it is preferable that the detection of the region of interest and a message M1 related to the movement guidance direction are displayed together. Further, as shown in FIG. 16, a message M2 for guiding the region of interest to the specific region may be displayed on the augmented display 18 as the movement notification information. The message M2 is a message that prompts a user to operate the endoscope 12 so that the region of interest ROI is put into the specific region 64 displayed on the augmented display 18 by a broken line. In FIG. 16, four L-shaped figures are displayed to surround the region of interest ROI as the region notification information. The augmented display 18 corresponds to a “display” of the present invention.

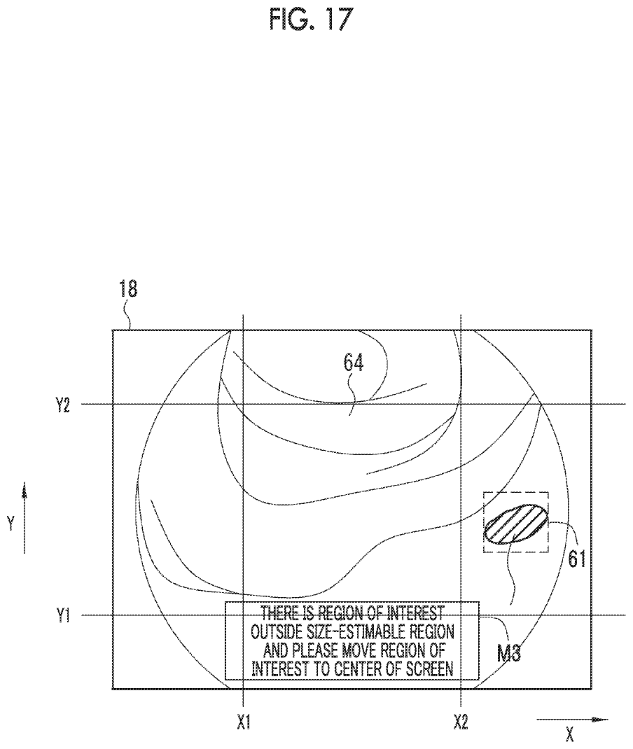

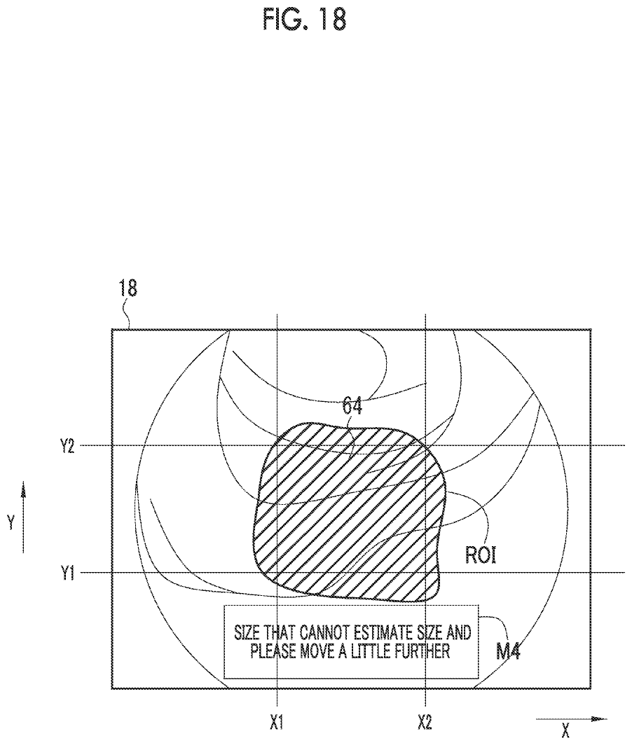

In a case where the size of the region of interest is not estimated, the second notification unit 57 gives a non-estimable notification notifying that the size cannot be estimated. It is preferable that the non-estimable notification is given using a display in the subject image or a voice. Specifically, in a case where the position of the region of interest is present outside the specific region 64 as shown in FIG. 17, the display of a message M3 notifying that the region of interest is present outside a size-estimable region is used as the non-estimable notification. User guidance for causing the region of interest to be put into the size-estimable region is included in the message M2. Further, in a case where the size of a region of interest is larger than the size of the specific region 64 as shown in FIG. 18, the display of a message M4 notifying that the size of the region of interest is a size which cannot be estimated is used as the non-estimable notification. User guidance for allowing a size to be estimated is included in the message M4.

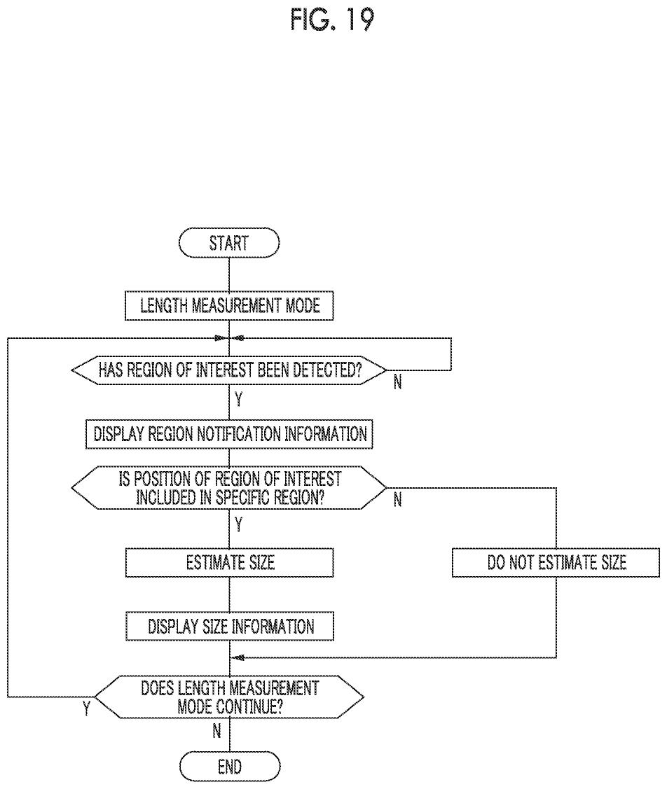

Next, a series of flows in the length measurement mode will be described with reference to a flowchart shown in FIG. 19. A user operates the user interface 16 to switch a mode to the length measurement mode. After a mode is switched to the length measurement mode, processing of detecting a region of interest from a subject image is performed. In a case where a region of interest ROI is detected, region notification information 61 is displayed on the region of interest ROI on the augmented display 18 to notify that the region of interest ROI is detected.

In a case where the position of the region of interest ROI is included in the specific region 64, the size of the region of interest ROI is estimated. After the estimation of the size of the region of interest ROI is completed, size information 62 is displayed on the region of interest ROI on the augmented display 18. On the other hand, in a case where the position of the region of interest ROI is not included in the specific region 64, the size of the region of interest ROI is not estimated. A series of processing described above is repeatedly performed while the length measurement mode continues.

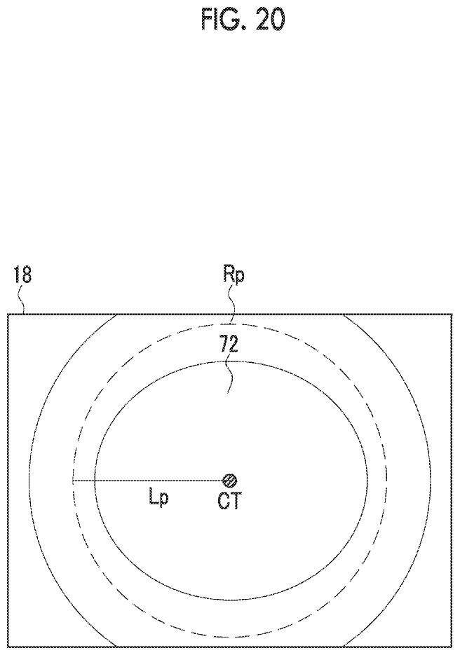

The specific region 64 is a rectangular region (see FIG. 8 and the like) in the embodiment described above, but the specific region may be included in a region Rp that is within a range of a certain distance Lp from a center CT of a subject image as shown in FIG. 20. For example, as shown in FIG. 20, an oval region included in the region Rp may be set as a specific region 72. The specific region may be a circular region.

In the embodiment described above, the hardware structures of processing units, which perform various types of processing, such as the reception unit 38, the signal processing unit 39, the display controller 40, the system controller 41, the static image storage unit 42, the data transmission/reception unit 43, the data transmission/reception unit 44, the signal processing unit 45, and the display controller 46 (including various controllers or processing units provided in these controllers and the like, are various processors to be described below. Various processors include: a central processing unit (CPU) that is a general-purpose processor functioning as various processing units by executing software (program); a programmable logic device (PLD) that is a processor of which the circuit configuration can be changed after manufacture, such as a field programmable gate array (FPGA); a dedicated electrical circuit that is a processor having circuit configuration designed exclusively to perform various types of processing; and the like.

One processing unit may be formed of one of these various processors, or may be formed of a combination of two or more same kind or different kinds of processors (for example, a plurality of FPGAs, or a combination of a CPU and an FPGA). Further, a plurality of processing units may be formed of one processor. As an example where a plurality of processing units are formed of one processor, first, there is an aspect where one processor is formed of a combination of one or more CPUs and software as typified by a computer, such as a client or a server, and functions as a plurality of processing units. Second, there is an aspect where a processor fulfilling the functions of the entire system, which includes a plurality of processing units, by one integrated circuit (IC) chip as typified by System On Chip (SoC) or the like is used. In this way, various processing units are formed using one or more of the above-mentioned various processors as hardware structures.

In addition, the hardware structures of these various processors are more specifically electrical circuitry where circuit elements, such as semiconductor elements, are combined. Further, the hardware structure of the storage unit is a storage device, such as a hard disc drive (HDD) or a solid state drive (SSD).

### EXPLANATION OF REFERENCES

10: endoscope system
12: endoscope
12a: insertion part
12b: operation part
12c: bendable part
12d: distal end part
12f: observation mode selector switch
12g: static image-acquisition instruction switch
12h: zoom operation part
13: light source device
14: processor device
15: display
16: user interface
17: augmented processor device
18: augmented display
22: illumination optical system
22a: illumination lens
23: imaging optical system
23a: objective lens
23b: zoom lens
30: light source unit
31: light source processor
32: imaging element
33: imaging controller
34: CDS/AGC circuit
35: A/D converter
36: communication I/F
37: communication I/F
38: reception unit
39: signal processing unit
40: display controller
41: system controller
42: static image storage unit
43: data transmission/reception unit
44: data transmission/reception unit
45: signal processing unit
46: display controller
50: region-of-interest detector
51: size estimation unit
52: first notification unit
53: size estimation controller
54: specific region setting unit
55: optical information acquisition unit
56: observation distance acquisition unit
57: second notification unit
61: region notification information
62: size information
64, 64a to 64e, 72: specific region
65: endoscope information storage memory
66: endoscope information acquisition unit
68: observation distance measurement unit
69: distance-measuring laser emitting unit
70: movement guidance direction
Ax: optical axis
CT: center
LG: light guide
Lm: distance-measuring laser
Lp: certain distance
M1 to M4: message
PS: subject image
Rp: region

What is claimed is:

1. An endoscope system comprising:
one or more processors configured to:
detect a region of interest in a subject image; and
perform a control to estimate a size of the region of interest in a case where a position of the region of interest in the subject image is included in a specific region, and not to estimate the size in a case where the position of at least the region of interest is not included in the specific region.

2. The endoscope system according to claim 1,
wherein the one or more processors are configured to set a position, a size, or a range of the specific region using optical information included in an imaging optical system used for acquisition of the subject image.

3. The endoscope system according to claim 2,
wherein the one or more processors are configured to receive endoscope information about an endoscope, and specifies the optical information based on the endoscope information.

4. The endoscope system according to claim 1,
wherein the one or more processors are configured to set a position, a size, or a range of the specific region using an observation distance indicating a distance to the region of interest.

5. The endoscope system according to claim 4, further comprising:
an endoscope that emits distance-measuring laser such that the distance-measuring laser intersects with an optical axis of an imaging optical system used for acquisition of the subject image,
wherein the one or more processors are configured to measure the observation distance from an irradiation position of the distance-measuring laser in the subject image.

6. The endoscope system according to claim 1,
wherein the one or more processors are configured to set a position, a size, or a range of the specific region using both optical information included in an imaging optical system used for acquisition of the subject image and an observation distance indicating a distance to the region of interest.

7. The endoscope system according to claim 1,
wherein the one or more processors are configured to notify a user of either detection of the region of interest or estimation of the size of the region of interest.

8. The endoscope system according to claim 1,
wherein the one or more processors are configured to give a movement guidance notification notifying a user of a direction in which the region of interest is moved to be included in the specific region in a case where the position of the region of interest is not included in the specific region.

9. The endoscope system according to claim 1,
wherein the one or more processors are configured to give a non-estimable notification notifying that the size cannot be estimated, in a case where the position of the region of interest is not included in the specific region or in a case where the size of the region of interest is larger than a size of the specific region so that the size is not estimated.

10. The endoscope system according to claim 9,
wherein the non-estimable notification is given using either a display in the subject image or a voice.

11. The endoscope system according to claim 1,
wherein the specific region is included in a region that is within a range of a certain distance from a center of the subject image.

12. The endoscope system according to claim 11,
wherein in a case where a first axis extending in a first direction and a second axis extending in a second direction orthogonal to the first direction are defined in the subject image, the specific region is a rectangular region that is surrounded by a first lower limit boundary line indicating a lower limit on the first axis, a first upper limit boundary line indicating an upper limit on the first axis, a second lower limit boundary line indicating a lower limit on the second axis, and a second upper limit boundary line indicating an upper limit on the second axis.

13. The endoscope system according to claim 11,
wherein the specific region is either a circular or an oval region.

14. The endoscope system according to claim 1,
wherein the one or more processors are configured to display the specific region on a display.

15. A method of operating an endoscope system including one or more processors, the method comprising following steps, performed by the one or more processors, of:
detecting a region of interest in a subject image; and
performing a control to estimate a size of the region of interest in a case where a position of the region of interest in the subject image is included in a specific region, and not to estimate the size in a case where the position of at least the region of interest is not included in the specific region.
